(19) United States
(12) Patent Application Publication
(10) Pub. No.: US 2004/0086946 A1
(43) Pub. Date: May 6, 2004

(54) Methods of isolation of active compounds and activated targets

(76) Inventors: Andrew J. Murphy, Croton-On-Hudson, NY (US); Y. Gopi Shanker, New York, NY (US); George D. Yancopoulos, Yorktown Heights, NY (US)

Correspondence Address: REGENERON PHARMACEUTICALS, INC, 777 OLD SAW MILL RIVER ROAD, TARRYTOWN, NY, 10591, US

(21) Appl. No.: 10/463,016

(22) Filed: Jun. 17, 2003

Related U.S. Application Data
(60) Provisional application No. 60/423,767, filed on Nov. 5, 2002.

(51) Int. Cl. G01N 33/53

(52) U.S. Cl. 435/7.1

(57) ABSTRACT

Methods for identifying a compound capable of modulating activity of a target active domain, by generating a first fusion protein having an anchor component and a variable component, generating a second fusion protein having a docking domain and an active domain, wheren the anchor component and the docking domain are binding partners, then contacting the fusion proteins under conditions in which the anchor component and the docking domain bind, and determining the activity of the target domain.

### STATEMENT OF RELATED APPLICATIONS

This application claims priority under 35 USC § 119(e) to provisional application U.S. Ser. No. 60/423,767 filed 5 Nov. 2002, which application is herein specifically incorporated by reference in its entirety.

### BACKGROUND OF THE INVENTION

1. Field of the Invention

This invention is related to methods for identifying and isolating active compounds as well as constitutively activated targets. Active compounds identified by the method of the invention are useful as potential therapeutic agents.

2. Statement of Related Art

Certain ligand molecules, such as some hormones, are known to contain separate domains responsible for activation (“message”) and binding (“address”). Prior art has modified low affinity agonists derived from message alone into high affinity molecules (both agonists and antagonists) (see, for example, Kawai et al. (1991) J. Med. Chem. 34:2068-2070; Wong et al. (1998) J. Med. Chem. 41:3417-3425).

The thrombin receptor contains an intrinsic agonist within its extended N-terminus which is not exposed until thrombin cleavage, which results in a new N-terminus that serves as a tethered ligand agonist for the receptor (Vu et al. (1991) Cell 64:1057-68). The tethered agonist also acts intermolecularly to activate nearby thrombin receptors (Chen et al. (1994) J. Biol. Chem. 269:16041-5). This interaction was exploited in a screening method to identify peptide agonists capable of activating the thrombin receptor (Chen et al. (1995) J. Biol. Chem. 270:23398-23401).

### SUMMARY OF THE INVENTION

The present invention utilizes the features of the message:address model by creating an artificial means of “addressing” potential messages to a target molecule. The message domain of a naturally occurring agonist or a test compound having low affinity to a target can be converted to a high affinity agonist or antagonist by appropriate addressing. As described more fully below, the present invention encompasses target molecules composed of a docking domain and an active domain, and a potentially active compound composed of an anchor component capable of binding to the docking domain and a potentially active or test component. Further included are screening methods for the identification and isolation of active compounds and constitutively activated targets. These methods can be directly applied to the rapid screening of libraries of potential active components.

In a first aspect, the invention features a method for identifying a compound capable of modulating activity of a target active domain, comprising (a) generating a first fusion protein, wherein the first fusion protein comprises an anchor component and a variable component; (b) generating a second fusion protein, wherein the second fusion protein comprises a docking domain and a potentially active domain, wherein the anchor component of the first fusion protein and the docking domain of the second fusion protein are binding partners; (c) contacting the first and second fusion proteins under conditions in which the anchor component and the docking domain bind; wherein the binding of the anchor component and docking domain do not affect the activity of the target domain; (d) determining the activity of the target domain relative to the activity of the target domain in the absence of the first fusion protein, wherein increased or decreased activity of the target domain in the presence of the first fusion protein indicates that the variable component of the first fusion protein is a modulator of the target domain. In one embodiment, the method is carried out with a library of first fusion proteins comprising the same anchor component and different variable components. The method of the invention may be conducted in vitro or in vivo, e.g., in an intact cell, the method of the invention may be used to identify an activator or inhibitor of the target active domain.

In specific embodiments, the binding partners are selected from a group consisting of (i) the Fc portion of an immunoglobulin and the Fc-binding portion of an Fc receptor; (ii) a protein domain and a antibody specific for the protein domain; (iii) a small molecule and a protein domain capable of binding the small molecule (iv) the Fc portion of an immunoglobulin and protein A or protein G; (v) a ligand and the ligand-binding domain of its cognate receptor; (vi) a pair of interacting leucine zippers; and (vii) fos and jun. Preferably, the binding affinity of the binding partners is at least 1 μM. In another embodiment, the binding partners bind to each other with an affinity at least 10 times higher than the variable component and active domain. In more specific embodiments, the protein domain and small molecule capable of binding the protein domain are selected from the group consisting of (i) a small molecule and a single-chain or multi-chain antibody immunospecific for the small molecule, (ii) fluorescein and an anti-fluorescein single-chain or multi-chain antibody; (iii) dinitrophenyl (DNP), or a DNP derivative and an anti- DNP single-chain or multi-chain antibody; (iv) novobiocin or a novobiocin derivative and a novobiocin-binding domain of gyrase B; (v) biotin, or a biotin derivative and avidin, streptavidin or neutravidin; (vi) FK506, or an FK506 derivative, and FKBP.

In specific embodiments, the activity of the active domain is determined by a means selected from the group consisting of signal transduction, signal transduction inhibition, a change in the level of cAMP, a calcium flux, a change in cell migration, the phosphorylation state of an indicator molecule, the rate of transcription of a reporter gene, channel dilation, ion gate opening or closure, change in extracellular or intracellular pH, translocation of a molecule within the cell, apoptosis, change in cell growth or change in metabolism.

The active domain may be any protein or protein fragment having an activity that can be modulated. More specifically, the active domain may be a receptor (such as a G-protein coupled receptor) (“GPCR”), an ion channel, an enzyme, a transporter, or a portion of any one of these targets. In specific embodiment, the active domain is isolated or present in a complex mixture; and/or present in solution, in a biological membrane or affixed to a surface. The active domain may be a monomeric, multimeric, or a non-protein macromolecule.

The docking domain is a component capable of binding a binding partner, termed an “anchor component”, and includes a protein such as a receptor. Further, the docking domain may be monomeric or multimeric.

In a third aspect, the invention provides a nucleic acid construct encoding a target fusion (chimeric) protein (“target protein”) comprising a docking domain and an active domain. The docking and active domains may be obtained, derived, and/or modified from naturally occurring proteins or protein fragments. In a second related aspect, the invention provides a target fusion protein encoded by the nucleic acid construct of the invention, comprising a docking domain and an active domain. The active domain may be covalenfly or noncovalently bound to the docking domain. In specific embodiments, the docking domain is ionically bound to another component, or is bound by intermolecular forces, e.g., including but not limited to H-bonding and Van der Waals forces.

In a fourth aspect, the invention provides a nucleic acid construct encoding a fusion (chimeric) protein, termed an “potentially active compound” comprising an anchor component and a potentially active (test or variable) component.

In a related fifth aspect, the invention features a potentially active compound comprising an anchor component and a potentially active (test or variable) component. The anchor component of the compound binds the docking domain of a target protein, thus delivering the active or test component to a desired target molecule with greater affinity than the binding of the active component alone to the active domain. The anchor or potentially active component may be obtained, derived, and/or modified from a naturally occurring or synthetic protein, peptide, or fragment thereof. Further encompassed by the invention are libraries of potential active compounds. Such libraries may be constructed by standard techniques known in the art. These techniques may include chemical synthesis, including combinatorial chemistry, or biological synthesis, including recombinant DNA technology or natural product synthesis.

In various embodiments, the test or variable component is connected to the anchor component by covalent forces, ionically, or by intermolecular forces. In a specific embodiment, the test or variable component is active, e.g., it is known to activate the active domain of the target molecule to some degree. In another specific embodiment, the ability of the test or variable component to activate the target molecule is not known.

The anchor component may be a protein, peptide, or molecule capable of binding to the docking domain. The anchor component may be monomeric or multimeric. Non-limiting examples of anchor components include any ligand, agonist, antagonist, antibody, or peptide that binds the docking domain.

The variable or test component may be a small molecule; a peptide agonist, antagonist, inhibitor, or activator; or any portion of a protein to be tested for affecting activity of the active target and/or inducing a physiological change. The variable compound may be monomeric or multimeric in composition.

The ability of a test or variable compound to activate the target molecule may be determined by a variety of methods known in the art. For example, determination of an activation reaction may be measured by physiological changes such as signal transduction, signal transduction inhibition, channel dilation, ion gate open/closure, cellular uptake or release of a solute, inhibition of cellular uptake or release of a solute etc.

In a sixth aspect, the invention features a method for identifying a compound capable of modulating activity of a target active domain, comprising (a) generating an anchor molecule comprising an anchor component and a variable component; (b) generating a target molecule comprising a docking domain and a potentially active domain, wherein the anchor component of the anchor molecule and the docking domain of the target molecule are binding partners; (c) contacting the anchor and target molecules under conditions in which the anchor component and the docking domain bind; wherein the binding of the anchor component and docking domain do not affect the activity of the potentially active domain; (d) determining the activity of the target domain relative to the activity of the target domain in the absence of the anchor molecule, wherein increased or decreased activity of the target domain in the presence of the anchor molecule indicates that the variable component of the anchor molecule is a modulator of the target domain. In this aspect of the invention, the variable component is a small molecule.

In a seventh aspect, the invention features an anchor molecule comprising an anchor molecule connected to a small molecule.

In an eighth aspect, the invention provides a method for converting a low affinity active compound into a high affinity active compound, comprising fusing a low affinity active compound to an anchor component. The resulting fusion compound exhibits an enhanced affinity for a desired target molecule.

In a ninth aspect, the invention provides a method for converting a low affinity target into a high affinity target, comprising fusing a low affinity active compound to a docking component. The resulting fusion protein exhibits an enhanced target affinity.

In a tenth aspect, the invention provides a transgenic non-human organism containing a nucleic acid construct of the invention. In a specific embodiment, the organism comprises a target molecule produced by the method of the invention. In a more specific embodiment, the organism is a knock-in for a target molecule of the invention. In an even more specific embodiment, the knock-in animal comprises a constitutively active target molecule. The transgenic knock-in animals of the invention are useful in a variety of ways, including for study of genotypic and/or phenotypic variation.

In an eleventh aspect, the invention features a library of anchored molecules, wherein each anchored molecule comprises a constant anchor component and a variable component, wherein the anchor component is capable of binding a target molecule without modulating activity of the target molecule. In a specific embodiment, the variable component is a small molecule.

In a twelfth aspect, the invention features a method of identifying a constitutively activated target molecule, the method comprising (a) constructing a fusion molecule comprising a variable or test compound fused to an active target domain; and (b) measuring the activity of the fusion molecule, wherein a fusion molecule exhibiting an increased activity relative to the active target domain is a constitutively activated target molecule. In a specific embodiment, the method is used to screen a library of fusion molecules. More specifically, the library of fusion molecules comprises a random peptide fused to a target domain, and the method identifies a random peptide capable of constitutively activating the target domain. Activation of the target domain may be determined by any method known to the art, including those listed above.

In a thirteenth aspect, the invention features a method for identifying a compound capable of binding a target docking domain, comprising (a) generating a first fusion protein comprising a test component and an active component; (b) generating a second fusion protein comprising a docking domain and an active domain, wherein the active component of the first fusion protein binds the active domain with low affinity; (c) contacting the first and second fusion proteins; (d) determining the activity of the active domain relative to the activity of the active domain in the absence of the first fusion protein, wherein increased or decreased activity of the active domain in the presence of the first fusion protein indicates that the test component of the first fusion protein is capable of binding the docking domain. This method of the invention, termed an “extracellular 2-hybrid screen” is useful for identifying a ligand capable of binding the docking domain, for example, when the docking domain is an orphan receptor with no known ligand. In one embodiment, the anchor and active components of the first fusion protein and/or the docking and active domains of the second fusion protein are connected via a spacer. In a more specific embodiment, the spacer is 1-15 amino acids; more specifically, the component elements are connected via a spacer that is 10-15 amino acids.

Other objects and advantages will become apparent from a review of the ensuing detailed description.

### BRIEF DESCRIPTION OF THE FIGURES

FIG. 1 is a pictorial illustration of a conventional method of screening for modulators of a target (A) and specific embodiments of the method of the invention (B, C).

FIG. 2 shows the dose response of HFRW-Fc (A) and MSH-Fc (B) on MC4R (Δ) and FcR-MC4 (□).

FIG. 3 illustrates constitutive activity of MSH-MC4 and HFRW-MC4 fusion proteins.

FIGS. 4A-B shows the screening a library of fusion proteins containing random 5 amino acid peptides fused at the amino terminus to the MC4R target molecule. A. Ninety four members of the library, as well as the unfused MC4 (first bar) and HFRW-MC4 fusion (second bar) were tested for constitutive activity after transfection into cells. B. Duplicate samples (minipreps A and B) of each positive were retested in the same manner. The sequence of the five amino acid peptides is shown.

FIGS. 5A-B shows the dose response of anchored and non-anchored small molecule agonists on scFv-P2Y6.

FIGS. 6A-B shows inhibition of MC4R upon activation by either natural or anchored agonists.

FIG. 7 provides the results of a GPCR-based extracellular 2-hybrid screen in which fusion proteins alpha1-MC4R, alpha2-MC4R, OGH-MC4R and hCG-MC4R are co-expressed with no compound (control)(no ligand), HFRW-alpha1, HFRW-alpha2, HFRW-OGH and HFRW-hCG. Activity of MC4R was measured with a luciferase reporter.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

General Description

Generally, the invention in part uses the binding interaction of a docking domain and an anchor component to bring a test or active component attached to the anchor into spatial proximity with an active domain attached to the docking domain. This interaction can be exploited to modulate the active domain, or to identify compounds capable of modulating the active domain.

The invention provides methods of identifying and isolating active compounds. The screening methods of the invention include a target molecule and a potentially active compound. The target molecule includes two parts, a docking domain and an active domain. The potentially active compound includes two parts, an anchor component and a potentially active component. The docking domain of the target molecule binds the anchor component of the active compound and facilitates the interaction between the active domain of the target molecule and the active component of the active compound. The method involves identification of an active component which affects in some way the activity of the target. The field also encompasses the use of this method to screen a library of potential active components, such that the amino acid and/or DNA sequences encoding the particular active component may be identified and utilized in further development. In particular, the method allows identification of activating ligands, agonists, antagonists, or portions thereof.

References herein to molecules, super-molecular complexes, proteins or entities are taken to mean either individual single molecules, super-molecular complexes, proteins or entities or mixtures or solutions containing many such molecules, super-molecular complexes, proteins or entities. As a non-limiting example, the term compound may refer to a single molecule of the compound or a mixture containing 1 nmole (approximately 6×10¹⁴) of molecules, or any other number of molecules, of the compound.

Anchor and Docking Binding Partners

The term “binding partners” is applied to the interaction between the docking domain and the anchor component. These components are selected based on their ability to interact with high specificity and high affinity. In one embodiment, the binding partners bind with an affinity of at least 1 μM Kd. In another embodiment, the affinity of the binding partners is at least 10-fold greater than the affinity of the variable/test component for the active domain. Examples of binding partners suitable for use as docking and anchor elements include, but are not limited to (i) the Fc portion of an immunoglobulin and the Fc-binding portion of an Fc receptor; (ii) a protein domain and a antibody specific for the protein domain; (iii) a small molecule and a protein domain capable of binding the small molecule (iv) the Fc portion of an immunoglobulin and protein A or protein G; (v) a ligand and the ligand-binding domain of its cognate receptor; (vi) a pair of interacting leucine zippers; and (vii) fos and jun.

In specific embodiments of the anchor and docking binding partners of the invention, the binding partners include (i) a small molecule and a single-chain or multi-chain antibody immunospecific for the small molecule, (ii) fluorescein and an anti-fluorescein single-chain or multi-chain antibody; (iii) dinitrophenyl (DNP), or a DNP derivative and an anti-DNP single-chain or multi-chain antibody; (iv) novobiocin or a novobiocin derivative and a novobiocin-binding domain of gyrase B; (v) biotin, or a biotin derivative and avidin, streptavidin or neutravidin; (vi) FK506, or an FK506 derivative, and FKBP.

Libraries of Anchor Compounds

In specific embodiments of the invention, libraries of anchored compounds are generated containing anchor molecules having a constant anchor component attached to different variable components. The variable compound may be a small molecule; a peptide that serves as an agonist, antagonist, inhibitor, or activator; or any portion of a protein to be tested for affecting activity of the active target and/or inducing a physiological change. The variable compound may be monomeric or multimeric in composition.

Determining Modulation of an Active Domain

Non-limiting examples of activities that may be measured to determine modulation of an active domain in a target molecule include signal transduction, signal transduction inhibition, second messenger production, inhibition of second messenger production, channel dilation, ion gate open/closure, a cellular response, a chemical reaction, inhibition of a chemical reaction, an enzyme reaction, inhibition of an enzyme reaction or any other measurable or detectable response. The activity may be measured by PCR, Taqman PCR, phage display systems, gel electrophoresis, capilliary electrophoresis, a two hybrid assay, northern or western blot analysis, immunohistochemistry, ELISA, competitive ELISA, radio-immune assay (RIA), time-resolved fluorescence, resonance energy transfer (such as FRET or BRET), colorimetry, calorimetry, patch clamping, electrophysiology, electrical potential, electrical conductance, microphysiometry, Schlieren optics, surface plasmon resonance, a receptor internalization assay, a yeast assay, a melanophore assay, an oocyte assay, a translocation assay, an arrestin assay, a reporter gene assay, a luciferase assay, an aequorin assay, a beta-galactosidase assay, a glucuronidase assay, a phosphatase assay, a kinase assay, a fluorescence polarization assay, a genetic assay, a growth assay, a chemotaxis assay, an apoptosis assay, an uptake assay, a release assay, a chromate release assay, a drug sensitivity assay, a proliferation assay, a survival assay, an MTS assay, a vital dye, fluctuation analysis, a fluorimeter, a spectrophotometer, a luminometer, a colorimeter, a calorimeter, a fluorescence imaging plate reader (FLIPR), a genetic analyzer, Biacore, a microscope, a high-content screening system, a DNA sequenator, a fluorescence activated cell sorter (FACS), photographic film, X-ray film, a CCD camera, a digital camera, a conventional scintillation camera, a gamma camera, a rectilinear scanner, a PET scanner, a SPECT scanner, an MRI scanner, an NMR scanner, a mass spectrometer or an X-ray machine or other means. In addition, any imaging agent known in the art may be employed, for example, a radionucleotide or a chelate. The change in the target molecule's activity may also be detected by detecting a change in its interaction with one or more proteins (see, e.g. PCT International Publication No. WO 96/34099, published Oct. 31, 1996).

Potential Active Compounds

Active compounds may be identified from a library of potentially active compounds following measurement of target activity using a variety of screening methods known in the art. Such methods include, but are not limited to, statistical methods that compare quantitative measures of activity. Such comparisons may be made between samples in which a potentially active compound is incubated with a target and any roughly equivalent sample or collection of samples expected to lack activity. Some examples of samples expected to lack activity include, but are not limited to, a sample to which no potentially active compound has been added, a sample to which a compound known to be inactive has been added, a sample in which a critical co-factor required for activity has been omitted, a sample in which one of the components required for activity has been inactivated, an average of samples containing compounds, the bulk of which are expected to be inactive or only weakly active, or the same sample, an identical sample or a similar sample in which the time or conditions of incubation prevent or reduce activity. Measurements of activity may be ratiometric instead of absolute, for instance the ratio of fluorescence emission of a sample at measured at one wavelength to its emission at a second wavelength may be a measure of activity regardless of the absolute amount of fluorescence. In other assays known in the art, such as in genetic selection, measurements of activity may be self-expository. In such assays, the assay itself is able to identify active compounds. The method of the present invention may be combined with assays utilizing other methods designed as secondary confirmatory assays or as assays for specificity or assays against potentially deleterious effects.

Extracellular 2-Hybrid Screen

The invention includes methods for identifying a ligand to a protein with no known ligand, e.g., an orphan receptor. In this embodiment, the invention comprises a first fusion protein comprising a test component as the anchor component and an active component capable of binding the active target with low affinity; a second fusion protein comprising a docking domain, for example, an orphan receptor, and an active domain. When the first and second fusion proteins are contacted together, the ability of the test anchor component to bind the docking domain may be determined by activation of the target domain by the low affinity active component. In one example of this embodiment of the invention, the active domain is MC4R, and the active component is the low affinity MC4R agonist, HFRW (SEQ ID NO:1); the docking domain is an orphan receptor, such as ROR2, and the test anchor component is a potential ROR2 ligand. Binding of the test anchor component to the docking domain brings HFRW (SEQ ID NO:1) to MC4R and allows HFRW activation of MC4R. In specific embodiments of the fusion proteins, the components of each first and second fusion proteins are connected via a spacer 1-15 amino acids in length. In a more specific embodiment the anchor and active components of the first fusion protein and/or the docking and active domains of the second fusion protein are connected via a 10-15 amino acid spacer.

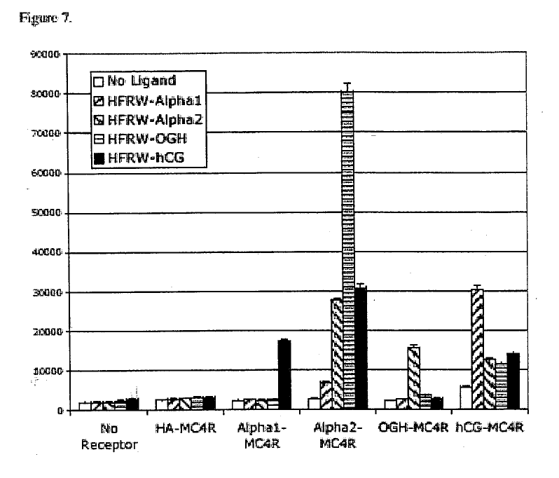

Another example of the extracellular 2-hybrid screening method of the invention utilizes glycoprotein hormone subunits. In one example, MC4R is used as the active domain, and HFRW (SEQ ID NO:1) as a known low affinity MC4R agonist. The docking domain is composed of one of four glycoprotein subunits (e.g., alpha1, alpha2, hCG, and OGH), and the anchor domain is one of the four glycoprotein subunits. The effect of co-expression of all first and second fusion protein combinations was measured by activation of MC4R (FIG. 7).

Transgenic Animals

The invention also relates to host cells and animals genetically engineered to express polypeptides or peptides including derivatives, fragments, or domains thereof, mutated, truncated or deletion forms thereof, fusion proteins, as well as host cells and animals genetically engineered to express the same. Animals of any species, including but not limited to mice, rats, rabbits, guinea pigs, pigs, goats, sheep, and non-human primates, may be used to generate transgenic or knock-in animals and their progeny, wherein “transgenic” means randomly integrated gene sequences from another source, as well as over-expressing endogenous sequences, and “knock in,” meaning the same except that such integration is targeted. Any technique known in the art may be used to introduce a transgene into an animal to produce a founder line of transgenic or knock-in animals, including pronuclear injection (U.S. Pat. No. 4,873,191); retroviral mediated gene transfer into germ lines (Van der Puttenn et al. (1985) Proc. Natl. Acad. Sci. USA 82:6148-6152); gene targeting in embryonic stem cells (Thompson et al. (1989)Cell 56:313-321); electroporation of embryos (Lo (1983) Mol. Cell Biol. 3:1803-1814); and sperm mediated gene transfer (Lavitrano et al. (1989) Cell 57:717-723). In addition, any technique may be used to produce transgenic or knock-in animal clones, for example nuclear transfer into enucleated oocytes of nuclei from cultured embryonic, fetal or adult cells induced to quiescence (Campbell et al. (1996) Nature 380:64-66). The invention provides for animals that carry the transgene in all of their cells as well as only some of their cells, for example, a particular cell type.

Specifilc Embodiments

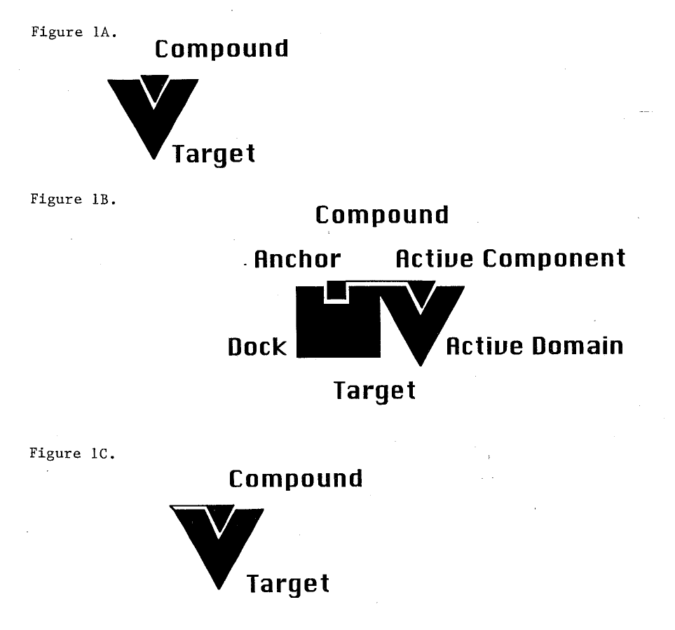

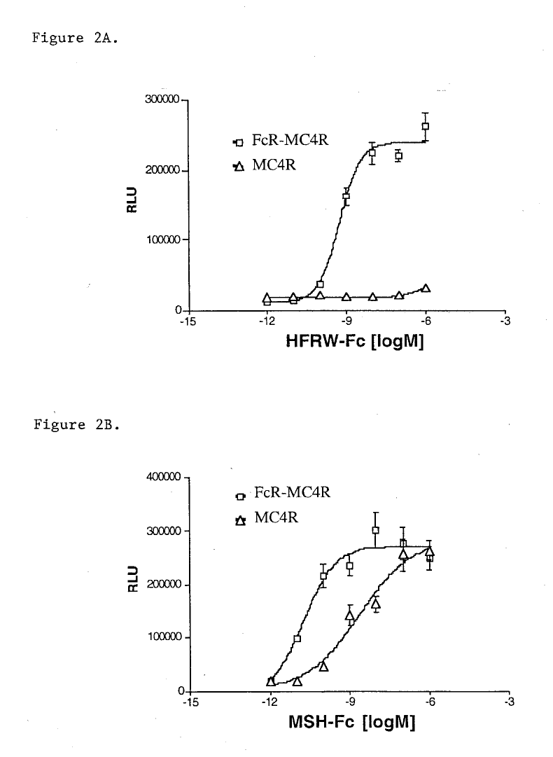

Example 1 describes construction of a target fusion protein in which the docking domain is the extracellular domain of a human Fc receptor (Fc-γR1) and the active domain is MC4R (FIG. 1B). Two anchor compounds were constructed having the HFRW peptide (SEQ ID NO:1) or the α-MSH peptide fused to the Fc anchor, and the ability of the anchored fusion proteins to stimulate the target molecules MC4R±Fc-γR1 measured (Example 2). Addition of increasing amounts of purified HFRW-Fc protein to HEK293 cells which had been transiently transfected with the FcR-MC4R construct resulted in a robust response that reaches a half maximal level at below 1 nM HFRW-Fc. In contrast, only a minute response was elicited when even the highest concentration of HFRW-Fc (1μM) was added to cells transfected with an MC4R construct lacking the FcR docking domain (FIG. 2). There appeared to be at least a 5 order of magnitude increase in potency of the HFRW peptide to activate MC4R by the addition of the Fc::FcR anchor::dock interaction.

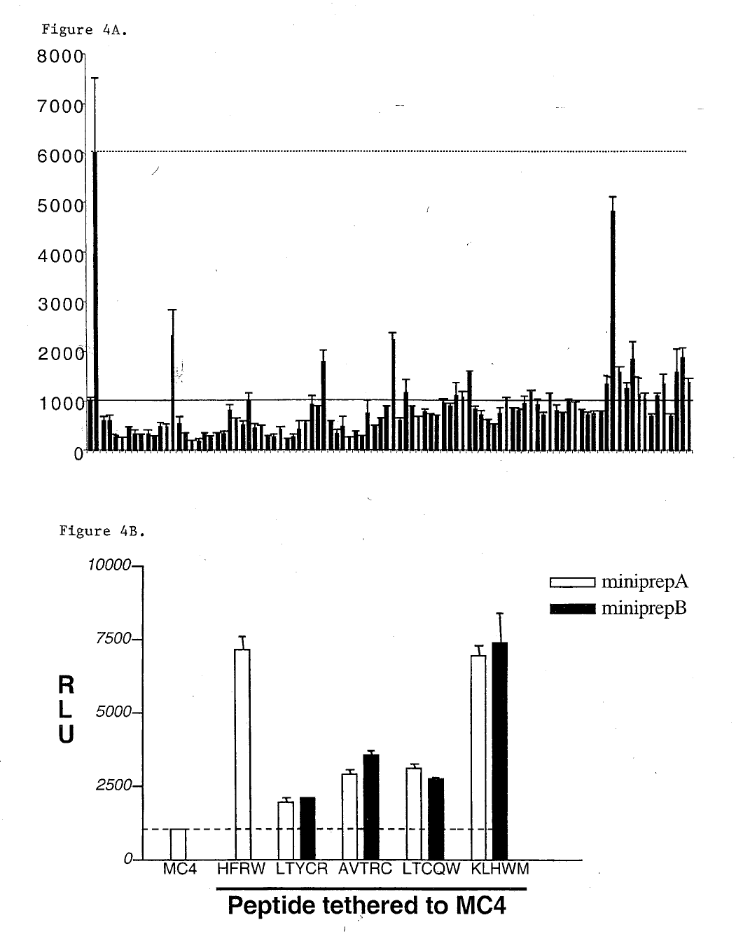

Example 2 describes one embodiment of the invention in which the binding of an active or test compound to its target molecule is potentiated by co-synthesis as part of a single protein. The ability of α-MSH-MC4R and HFRW-MC4R fusion proteins to constitutively activated MC4R was measured with a transcriptional reporter Cre-luficerase. A library of MC4R molecules having a random 5 amino acid sequence fused to the N-terminus of the MC4R sequence were screened for ability to constitutively activate MC4 (Example 3). Several molecules were identified as novel activators (or active compounds) of MC4 (FIG. 4).

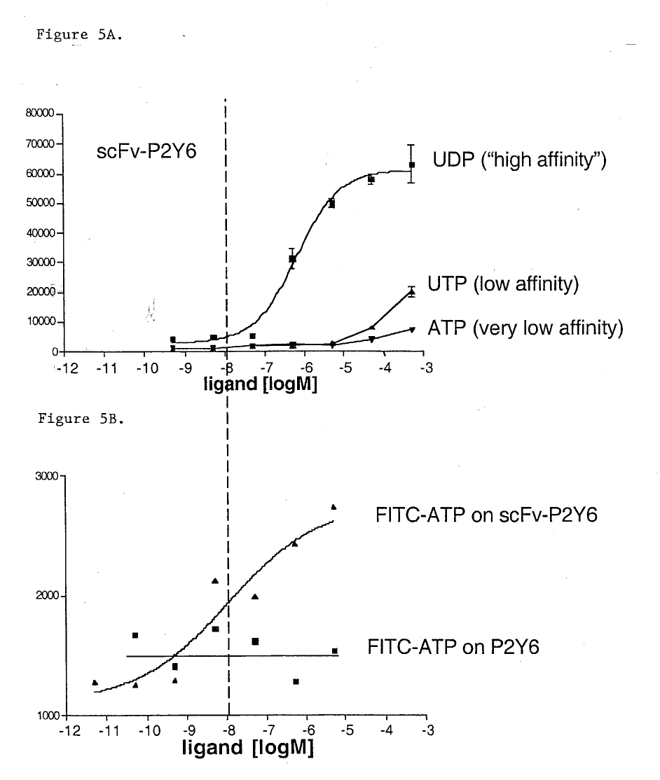

Example 4 shows the activation of a target fusion protein, scFv-P2Y6, by non-anchored small molecule agonists (UDP, UTP, and ATP) compared to activation with an anchored compound FITC-ATP (FIG. 5A-B). FITC-ATP was at least 10,000-fold more potent in activating P2Y6 than its un-anchored counterpart, ATP. FITC-ATP failed to activate a P2Y6 receptor construct which lacks an anti-fluorescein scFv docking domain at the doses tested (FIG. 5B).

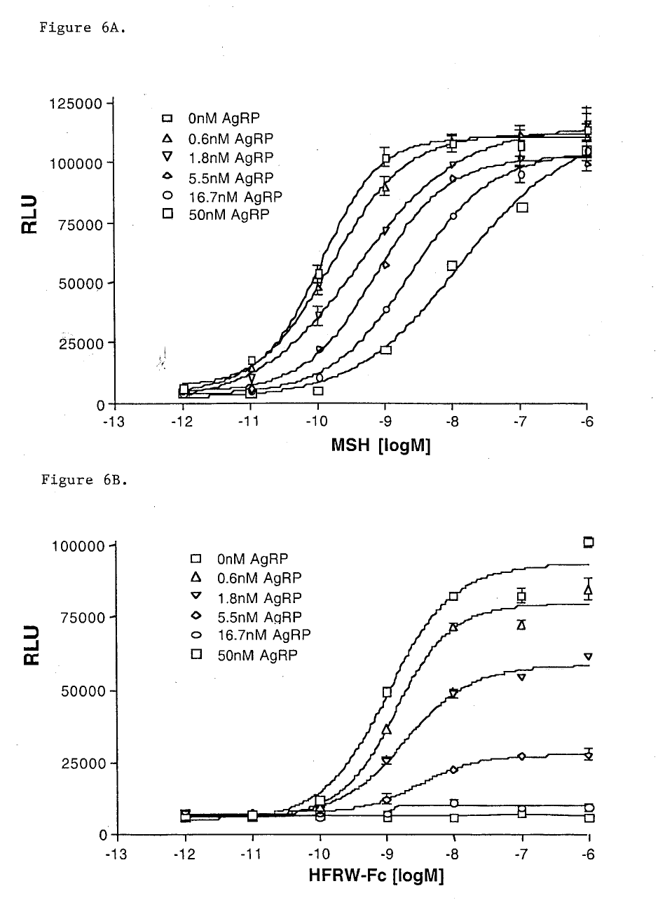

The effect of a known antagonist, agouti-related protein (AGRP), of the MC4 receptor was studied with the target FcR-MC4R fusion protein activated with either the natural agonist (α-MSH) or the anchored agonist (HFRW-Fc) (Example 5). Increasing amounts of AGRP shift the dose response curve of α-MSH progressively to the right without major changes in its shape or maximal stimulation level (FIG. 6A). In contrast, increasing amounts of AGRP were able to dramatically reduce the maximal stimulation levels obtained with HFRW-Fc (FIG. 6B).

The results described in Example 5 are highly advantageous for high throughput antagonist screening. Screening for antagonists is typically performed by incubating test compounds with the target followed by addition of a single dose of the activator or agonist. Inhibitor activity is then scored as a diminution of the level of activity. The dose of activator that is used needs to be carefully gauged so as to maximize both the reproducibility of the assay and its sensitivity to inhibitors. FIG. 6A illustrates these conflicting concerns present in prior art assays. Concentrations of an activator at or very near the saturation levels (>10 nM MSH) enhance reproducibility (less variation in stimulated levels with slight variation of MSH concentration) but reduce sensitivity (more AGRP is required to achieve a given level of inhibition). Concentrations of an activator closer to those which gives half maximal stimulation are more completely inhibited by lower concentrations of AGRP but slight variations of MSH concentration give very different stimulation levels greatly reducing reproducibility.

In contrast, both the maximal stimulation level as well as a constant inhibited level for each concentration of AGRP are consistently displayed over two logs of HFRW-Fc concentration (from 10 nM to 1 μM) (FIG. 6B). This represents a drastic improvement in reproducibility. In addition the percent inhibition is greater for each concentration of AGRP translating into an assay which is both more reproducible and more sensitive.

Examples 6 and 7 illustrate two different embodiments of the extracellular 2-hybrid screening method of the invention. Example 6 illustrates a method of identifying the ability of a test anchor component to bind to a known docking domain which does not have a known ligand, e.g., an orphan receptor such as ROR2. The ability of a fusion protein having a variety of test anchor domains to bind ROR2 is determined by activation of the active domain, e.g., MC4R, by a known low affinity agonist, e.g., HFRW (SEQ ID NO:1). Example 7 illustrates the extracellular 2-hybrid screen using glycoprotein hormone subunits. The target fusion protein is one of the four glycoprotein subunits and the active domain MC4R. Binding between the anchor and docking components was measured as an increase in MC4R activity (FIG. 7).

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Potentiation of Activation by Anchored Agonists of a Dock-Receptor Fusion Protein.

Alpha-MSH (alpha-melanocyte-stimulating hormone) is a 13 amino acid agonist for melanocortin receptor 4 (MC4 or MC4R). HFRW is a four amino acid (His Phe Arg Trp) (SEQ ID NO:1) peptide that is present within alpha-MSH as well as other melanocortin agonists, and that has been shown to fully activate melanocortin receptors, but only at very high concentrations.

A fusion protein gene was constructed between the extracellular domain (amino acid residues 1-292) of a human Fc receptor (Fc-gammaR1=CD64; NM_{—}000566) and the human MC4 receptor. This was done by PCR amplifying the Fc-gammaR1 extracellular domain and HA-tagged human MC4 receptor coding regions in two separate reactions. The primers used for the PCR reactions were: Hind-FcRF (FcR forward primer): 5′ GGGAAGCTTCCACCATGTGGTTCTTGACAACTCT 3′ (SEQ ID NO:2); FcR-ECDrev (reverse primer for FcR): 5′ AGGGATAGGATCCATGAAACC AGACAGGAGTTGG 3′ (SEQ ID NO:3); HA-MC4R-F: (Forward primer for HA-MC4R): 5′ GGTTTCATGGATCCTATCCCTATGACGTCCCGG 3′ (SEQ ID NO:4); Xho-HA-MC4R-rev (reverse primer for HA-MC4R): 5′ AGACTCGAGCGGCCGCTTAATATCTGC 3′ (SEQ ID NO:5). The two PCR products were then “sewn” together in a second round of PCR using a 14 bp overlap in the sequences of FcR-ECDrev and HA-MC4R-F. The final PCR product was cloned into an expression vector. Experiments (not shown) demonstrated that the FcR-MC4R fusion protein, expressed in HEK293 cells, responded to MSH in a manner identical to the native MC4R.

Two fusion construct protein constructs were similarly constructed with Fc as the anchor component and the either HFRW peptide (SEQ ID NO:1) or α-MSH as the test component: HFRW-Fc and α-MSH-Fc. Proteins derived from the HFRW-Fc and αX-MSH-Fc expression constructs were purified from culture supernatants of transiently transfected CHO cells by affinity chromatography over protein-G columns and gel filtration. The purified fusion proteins were quantified by comparison of band intensities on stained SDS-PAGE gels to those of Fc standards.

HFRW-Fc (FIG. 2A) or MSH-Fc (FIG. 2B) were tested for the ability to activate a target molecule±a docking domain: MC4R alone, (Δ) or MC4R-FcγR1 ( ). MC4R was stimulated to half of its maximal level by 10,000-fold less of the anchored compound HFRW-Fc when it contains a docking domain (FIG. 1A). MC4R was stimulated to half of its maximal level by 100-fold less of the α-MSH when it was fused to an anchor component. Unrelated Fc containing proteins failed to activate the FcR-MC4R construct. Activation of MC4R was assayed 6 hours after compound addition using a co-transfected CRE-luciferase construct (pCRE-Luc; Stratagene) and measuring luminescence after lysis and addition of the luciferase substrate (Tropix Luciferase Assay Kit).

### Example 2

Constitutive Activity of MSH-MC4R and HFRW-MC4R Fusion Protein.

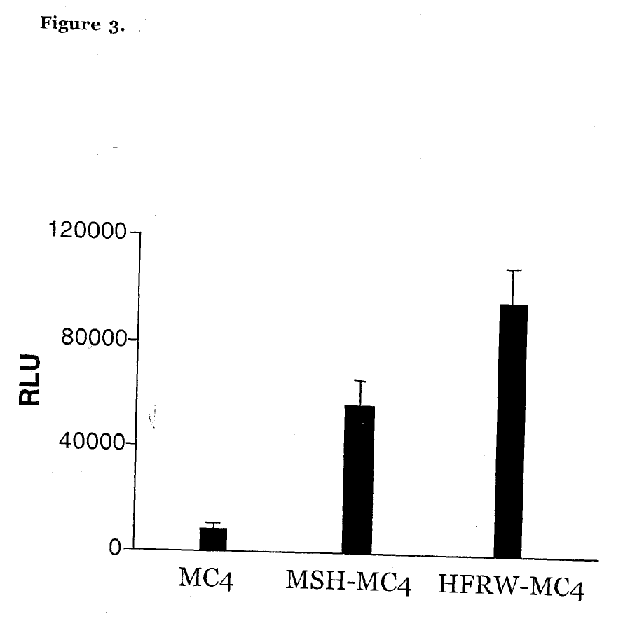

Constructs were built to express either an epitope tagged version of MC4R (MC4), a fusion protein between α-MSH and MC4R (MSH-MC4) or a fusion protein between the HFRW peptide and MC4R (IFRW-MC4). These constructs were co-transfected in triplicate with the CRE-luciferase reporter into HEK293 cells and two days later the cells were assayed for luciferase activity as described above. As shown in FIG. 3, both the MSH-MC4 fusion protein and the HFRW fusion protein show a significantly higher level of CRE-luciferase activity than the MC4 receptor on its own, and the HW-MC4 fusion showed as high or higher a level of activation as the α-MSH-MC4 fusion. In additional experiments (not shown) the level of activation of the α-MSH-MC4 fusion was found to be equivalent to the level of activation of an MC4 receptor in the presence of long term exposure to saturating amounts of α-MSH.

### Example 3

Construction and Screening of a Library of Randomer-MC4R Fusion Proteins.

A library in which five random amino acids were fused to the amino terminus of MC4R was constructed by PCR. Briefly, an oligonucleotide was designed that encoded each amino acid of a five amino stretch with an NNK (N corresponds to any of the four nucleotides and K corresponds to G or T) triplet followed by homology to the 5′end of the MC4 coding sequence. This oligonucleotide was used with a primer homologous to the 3′end of the MC4 coding sequence to generate a product that was subsequently ligated into an expression vector (a pCDNA 3.1 derivative) and transfected into E. coli. Individual colonies were expanded and the plasmids from several of these were sequenced to confirm that each encoded a different 5 amino acid sequence fused to MC4.

Several hundred members of the library were picked, and separate DNAs were prepared. These DNA preparations were co-transfected with a CRE-luciferase reporter into HEK293T cells, and luciferase levels were measured two days later as described above. FIG. 4A shows the results of assays on 94 library members compared to MC4 (first bar) and HFRW-MC4 (second bar). Hits in this assay, that is, those plasmids that conferred a higher level of luciferase activity, were re-prepared in duplicate and re-screened in the same manner (FIG. 4B).

### Example 4

Potentiation of the Activation of the P2Y6 Receptor by Anchored Small Molecule Agonists.

A fusion protein was constructed between a single chain antibody against fluorescein (Boder et al. (2000) Proc. Natl. Acad. Sci. USA 97:10701-5) (docking domain) and the human P2Y6 receptor (GenBank accession number: NP_{—}004145) (active domain). The target fusion protein, termed scFv-P2Y6, was co-transfected with the calcium-sensitive luminescent protein aequorin into HEK293 cells. After two days, the transfected cells were pre-loaded for two hours with the aequorin substrate coelenterazine (Molecular Probes) and then various amounts of UDP, UTP or ATP were added immediately followed by measurement of luminescence (FIG. 5A). The activation profile of these three agonists on the fusion protein closely mimicked their activation of the unfused P2Y6 receptor (not shown). UDP was the most potent activator; activation by UTP and ATP required much higher concentrations of these molecules. In contrast to ATP alone, an anchored compound, a fluorescein-ATP conjugate (FITC-ATP; Perkin-Elmer), was able to activate the scFv-P2Y6 receptor at low concentrations (FIG. 5B) with a half-maximal stimulation occurring at about 10 nM.

### Example 5

Improved Behavior of Anchored Agonist for Detecting Antagonists.

For this inhibition experiment, activating compounds (α-MSH and HFRW-Fc) were added 30 minutes after the inhibitor, AGRP. Activation of FcR-MC4R was assayed 6 hours after compound addition using a co-transfected CRE-luciferase construct (pCRE-Luc; Stratagene) and measuring luminescence after lysis and addition of the luciferase substrate (Tropix Luciferase Assay Kit). Results are shown in FIGS. 6A-B.

### Example 6

GPCR-Based Extracellular 2-Hybrid Screen.

Two fusion proteins are constructed as described above: the first with MC4R as an active domain, and the orphan receptor ROR2 as the docking domain, and a second fusion protein with potential ROR2 ligands as test anchor components and HFRW (SEQ ID NO:1) as the active component. The potential ROR2 ligands are encoded by a variety of experimental cDNA sequences. The first and second fusion proteins are contacted under conditions in which a test anchor component capable of binding the docking domain brings HFRW (SEQ ID NO:1) into close proximity with the active domain MC4R, resulting in activation of MC4R. The activation of MC4R by HFRW (SEQ ID NO:1) identifies a test anchor capable of binding ROR2.

### Example 7

Glycoprotein Hormone Subunit-Based Extracellular 2-Hybrid Screen.

Two sets of fusion proteins were constructed as follows: a set of target first fusion proteins with MC4R as an active domain and either one of four glycoprotein hormone subunits as the docking domain (alpha1, alpha2, hCG, and OGH) or, as a control, no docking domain (HA-MC4R); a set of second compound fusion proteins with each of the four glycoprotein hormone subunits as an anchor component and HFRW (SEQ ID NO:1) as the active component. A 15 amino acid spacer (GRAYPYDVPDYAGIL) (SEQ ID NO:6) including an HA epitope tag was included between each glycoprotein hormone subunit and MC4R, and a 13 amino acid spacer (GGGGSTGGGGGSG)(SEQ ID NO:7) was included between HFRW (SEQ ID NO:1) and each glycoprotein hormone subunit. Signal sequences were included at the beginning of each construct.

Results of experiments co-expressing the target and components fusion proteins are shown in FIG. 7. Each of the target fusion proteins (Alpha1-mc4r, Alpha2-MC4R, OGH-MC4R and HCG-MC4R) as well as controls (no receptor and HA-MC4R) were co-expressed with either no compound (“no ligand”, FIG. 7) or one of the four target fusion proteins (HFRW-Alpha1, HFRW-Alpha2, HFRW-OGH and HFRW-hCG) by co-transfection of HEK293 cells stably expressing a CRE-luciferace reporter. Two days after transfection, the activity of the target fusion protein was measured by assaying luciferase activity as relative light units (Y axis, FIG. 7) on a luminometer. Increased activity of the MC4R active domain relative to the “no ligand” control reflects binding between the anchor and docking components (glycoprotein hormone subunits). The results demonstrate that alpha2 binds to OGH, hCG, and itself, whereas alpha1 binds hCG.

What is claimed is:

1. A method for identifying a compound capable of modulating activity of a target active domain, comprising:
(a) generating a first fusion protein, wherein the first fusion protein comprises an anchor component and a variable component;
(b) generating a second fusion protein, wherein the second fusion protein comprises a docking domain and an active domain, wherein the anchor component of the first fusion protein and the docking domain of the second fusion protein are binding partners;
(c) contacting the first and second fusion proteins under conditions in which the anchor component and the docking domain bind; wherein the binding of the anchor component and docking domain do not affect the activity of the target domain; and
(d) determining the activity of the target domain relative to the activity of the target domain in the absence of the first fusion protein, wherein increased or decreased activity of the target domain in the presence of the first fusion protein indicates that the variable component of the first fusion protein is a modulator of the target domain.

2. The method of claim 1, wherein the first fusion protein is a library of fusion proteins comprising the same anchor component and different variable components.

3. The method of claim 1, wherein the binding partners are selected from a group consisting of (i) the Fc portion of an immunoglobulin and the Fc-binding portion of an Fc receptor; (ii) a protein domain and a antibody specific for the protein domain; (iii) a small molecule and a protein domain capable of binding the small molecule (iv) the Fc portion of an immunoglobulin and protein A or protein G; (v) a ligand and the ligand-binding domain of its cognate receptor; (vi) a pair of interacting leucine zippers; and (vii) fos and jun.

4. The method of claim 3, wherein the binding affinity of the binding partners is at least 1 μM.

5. The method of claim 3, wherein the binding partners bind to each other with an affinity at least 10 times higher than the variable component and active domain.

6. The method of claim 3, wherein The above method, wherein the protein domain and small molecule capable of binding the protein domain are selected from the group consisting of (i) a small molecule and a single-chain or multi-chain antibody immunospecific for the small molecule, (ii) fluorescein and an anti-fluorescein single-chain or multi-chain antibody; (iii) dinitrophenyl (DNP), or a DNP derivative and an anti- DNP single-chain or multi-chain antibody; (iv) novobiocin or a novobiocin derivative and a novobiocin-binding domain of gyrase B; (v) biotin, or a biotin derivative and avidin, streptavidin or neutravidin; (vi) FK506, or an FK506 derivative, and FKBP.

7. The method of claim 1, conducted in a cell.

8. The method of claim 1, wherein the activity of the active domain is determined by a means selected from the group consisting of signal transduction, signal transduction inhibition, a change in the level of cAMP, a calcium flux, a change in cell migration, the phosphorylation state of an indicator molecule, the rate of transcription of a reporter gene, channel dilation, ion gate opening or closure, change in extracellular or intracellular pH, translocation of a molecule within the cell, apoptosis, change in cell growth or change in metabolism.

9. The method of claim 1, wherein the compound identified is an activator and the activity of the target domain is increased in the presence of the first fusion protein.

10. The method of claim 1, wherein the active domain is selected from the group consisting of a membrane channel, a symporter transproter; an antiporter transporter; an ATPase; an enzyme; or a receptor.

11. The method of claim 10, wherein the receptor is a G-protein coupled receptor (GPCR).

12. A library of anchored fusion proteins, wherein each fusion protein comprises a constant anchor component and a variable component, wherein the anchor component is capable of binding a target molecule without modulating activity of the target molecule.

13. A method of identifying a constitutively activated target molecule, the method comprising:
(a) constructing a fusion protein comprising a variable or test compound fused to an active target domain; and
(b) measuring the activity of the fusion protein, wherein a fusion protein exhibiting an increased activity relative to the active target domain is a constitutively activated target molecule.

14. A library of fusion proteins, wherein each fusion protein comprises a variable compound fused to a target domain.

15. A method for identifying a compound capable of modulating activity of a target active domain, comprising:
(a) generating an anchor molecule comprising an anchor component and a variable component;
(b) generating a target molecule comprising a docking domain and a potentially active domain, wherein the anchor component of the anchor molecule and the docking domain of the target molecule are binding partners;
(c) contacting the anchor and target molecules under conditions in which the anchor component and the docking domain bind; wherein the binding of the anchor component and docking domain do not affect the activity of the potentially active domain,
(d) determining the activity of the target domain relative to the activity of the target domain in the absence of the anchor molecule, wherein increased or decreased activity of the target domain in the presence of the anchor molecule indicates that the variable component of the anchor molecule is a modulator of the target domain.

16. The method of claim 15, wherein the variable component is a small molecule.

17. A method of identifying a compound capable of binding a known protein, comprising:
(a) generating a first fusion protein comprising a test component and an active component;
(b) generating a second fusion protein comprising a docking domain and an active domain, wherein the active component of the first fusion protein binds the active domain with low affinity and wherein the docking domain is a known protein or fragment thereof;
(c) contacting the first and second fusion proteins; and
(d) determining the activity of the active domain relative to the activity of the active domain in the absence of the first fusion protein, wherein increased or decreased activity of the active domain in the presence of the first fusion protein indicates that the test component of the first fusion protein is capable of binding the docking domain.

18. The method of claim 17, wherein the test and active components and/or the active and docking domains are connected by a spacer of 1-15 amino acids.

19. The method of claim 18, wherein the spacer is 10-15 amino acids.

20. An assay kit for identifying a compound capable of binding a target active domain, comprising:
(a) a first fusion protein, wherein the first fusion protein comprises an anchor component and a variable component;
(b) a second fusion protein, wherein the second fusion protein comprises a docking domain and an active domain, wherein the anchor component of the first fusion protein and the docking domain of the second fusion protein are binding partners;
(c) means for measuring activity of the target domain; and
(d) instructions for conducting the assay.


SEQUENCE LISTING (7 sequences)

SEQ ID NO: 1 - protein, 4 aa, homo sapiens
HFRW

SEQ ID NO: 2 - DNA, 34 bp, homo sapiens
gggaagcttccaccatgtggttcttgacaactct

SEQ ID NO: 3 - DNA, 34 bp, homo sapiens
agggataggatccatgaaaccagacaggagttgg

SEQ ID NO: 4 - DNA, 33 bp, homo sapiens
ggtttcatggatcctatccctatgacgtcccgg

SEQ ID NO: 5 - DNA, 27 bp, homo sapiens
agactcgagcggccgcttaatatctgc

SEQ ID NO: 6 - protein, 15 aa, homo sapiens
GRAYPYDVPDYAGIL

SEQ ID NO: 7 - protein, 13 aa, homo sapiens
GGGGSTGGGGGSG